(19) United States
(12) Patent Application Publication
(10) Pub. No.: US 2022/0362048 A1
(43) Pub. Date: Nov. 17, 2022

(54) Body waste management system

(71) Applicant: Latifah Elizabeth Oliphant, New York, NY (US)

(72) Inventor: Latifah Elizabeth Oliphant, New York, NY (US)

(73) Assignee: Latifah Oliphant

(21) Appl. No.: 17/300,296

(22) Filed: May 17, 2021

(51) Int. Cl. A61F 5/44; A61F 13/45

(52) U.S. Cl. CPC A61F 5/4401; A61F 13/45; A61F 5/4404; A61F 2013/4506; A61F 2013/1513

(57) ABSTRACT

A body waste collection system for collecting excretions from the anus and/or urethra of female humans and animals. The body waste collection system includes a pad that has a central opening of a shape and shape that corresponds to a shape and size of a body orifice, wherein the pad can adhere to the skin area around the body orifice. A tubing fluidly connects the central opening of the pad to a waste collection bag, such as excretions from the body orifice can pass through the central opening into the tubing, and the tubing can direct the excretions into the waste collection bag.

### CROSS-REFERENCE TO RELATED APPLICATIONS

This application claims priority from the U.S. non-provisional patent application Ser. No. 15/932,651, filed on Apr. 2, 2018, which is incorporated herein by reference in its entirety.

### FIELD OF INVENTION

The present invention relates to a body waste collection system, and more particularly, the present invention relates to a waste collection system for persons suffering from bladder and/or bowel incontinence and animals.

### BACKGROUND

Certain medical conditions in persons can cause continuous or frequent discharge from the bowel or urinary bladder. This may be a temporary situation for different reasons and for different lengths of time. The medical condition can also become chronic and affects either bladder or bowel or both. Besides the medical condition, people in their late seventies may lose their control over bladder and/or bowel function resulting in incontinence problems.

persons suffering from bladder and/or bowel incontinence may lose confidence. Certain incidences of involuntary passing of urine or faces may become embarrassing for the persons. Moreover, the quality of life may be adversely affected in such persons.

Thus, a need is appreciated for a body waste collection system that can manage bladder and/or bowel incontinence in persons and allow them to live a life with dignity and pleasure.

The term “person(s)” herein after refers to person(s) suffering from bladder and/or bowel incontinence and includes animals.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a body waste collection system for persons suffering from bladder and/or bowel incontinence and animals.

It is another object of the present invention that the body waste collection system allows persons suffering from bladder and/or bowel incontinence to live with dignity.

It is still another object of the present invention that the body waste collection system can allow persons to attend events without anxiety.

It is yet another object of the present invention that the body waste collection system can prevent bedsores.

It is a further object of the present invention that the body waste collection system can prevent catheter-associated complications.

It is still a further object of the present invention that the body waste collection system can prevent any embarrassment associated with events of incontinence in public.

It is an additional object of the present invention that the body waste collection system provides a hygienic way to dispose of bodily wastes.

In one aspect, disclosed is a body waste collection system that may include tubing that has a proximal end and a distal end. The proximal end of the tubing can be flared to be an attachment point. The body waste collection system further includes one or two pads i.e., a urethral pad and/or a rectum pad, the pads can include a central opening that can be removably or integrally connected to the flared proximal end of the tubing. In one case, each of the two pads can have an opening that connects to a single flared proximal end of the tubing. In one case; the proximal end of the tubing can be bifurcated to two branch tubes, wherein each branch tube can have a flared end that can be connected to two openings of the two pads or two openings in a single pad. In one case, two tubes can be used for two pads. The distal end of one or two tubes can be fluidly connected to a waste collection bag.

In one aspect, only the urethral pad or the rectum pad can be provided. In one case, both the urethral pad and the rectum pad can be provided. In case, both the urethral pad and the rectum pad are provided, the urethral pad and the rectum pad can be separate or integral.

In one aspect, the pad can have a continuous flange along a periphery. To the flange can be applied a layer of adhesive that may permit the flange to adhere to the skin. The single pad can be applied to the skin area around a body orifice. In the case of the rectum pad, the rectum pad can be applied to the skin area around the anus. In the case of the urethral pad, the flange can be applied over and around the labia minora.

In one aspect, the shape and dimension of the pads including integral or separate urethral and rectum pads can be such as to cover the urethra and the anus. In one case, the opening in the rectum pad can be round to receive excretions from the rectum. In one case, the opening of the urethral pad can be oblong or rectangular to cover the labia minora.

In one aspect, the adhesive layer of the pad can be temporarily protected by a release liner. A user can remove the release liner and apply the pad around the anus or labia minora. The opening in the pad can be sealed to the proximal flared end of the tubing forming a passage. It is to be understood that the pad and the tubing can be integral or interchangeable. The pads can be applied either to the anus or labia minora or both to collect the bladder and/or bowel excretions, wherein the tubing can direct the excretions to the waste collection bag.

In one aspect, the tubing and the flared end of the tubing can be flexible and collapsible; and can expand readily for surges of expelled urine and feces.

In one aspect, the tubing can be made of a medical-grade rubber, plastic, silicon, and like material known to a skilled person for use in catheters.

In one aspect, the body waste collection system can be used in adults, children, and pets.

In one aspect, persons using the body waste collection system can rest, lay down, walk, sit, and stand, run, and can engage in physical activities. The pad and the flared end of tubing can be soft and collapsible making it comfortable to wear for a long duration.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows the body waste collection system having a urethral pad, a tubing, and a waste collection bag, according to an exemplary embodiment of the present invention.

FIG. 2 shows a rectum pad, according to an exemplary embodiment of the present invention.

FIG. 3 shows a pad having two openings for the anus and urethra, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

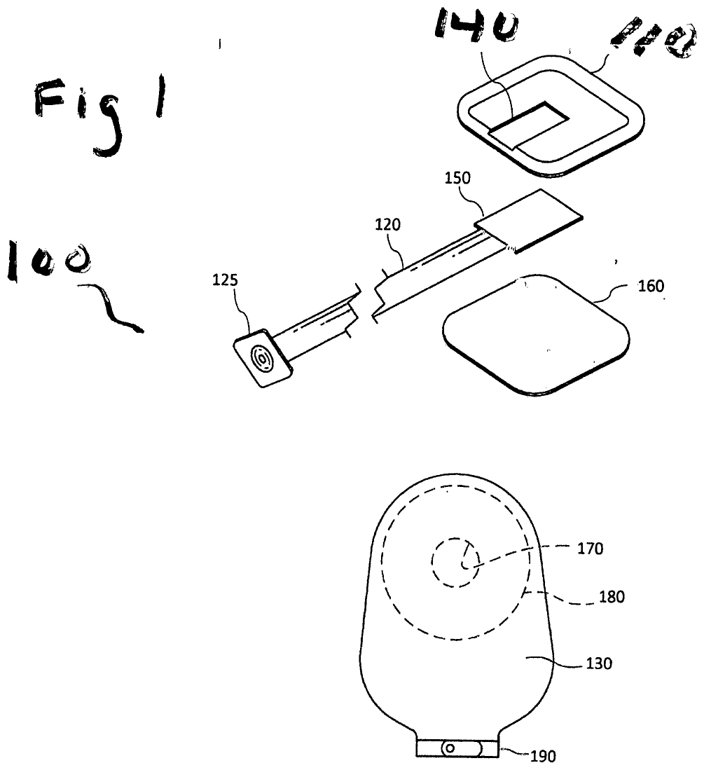

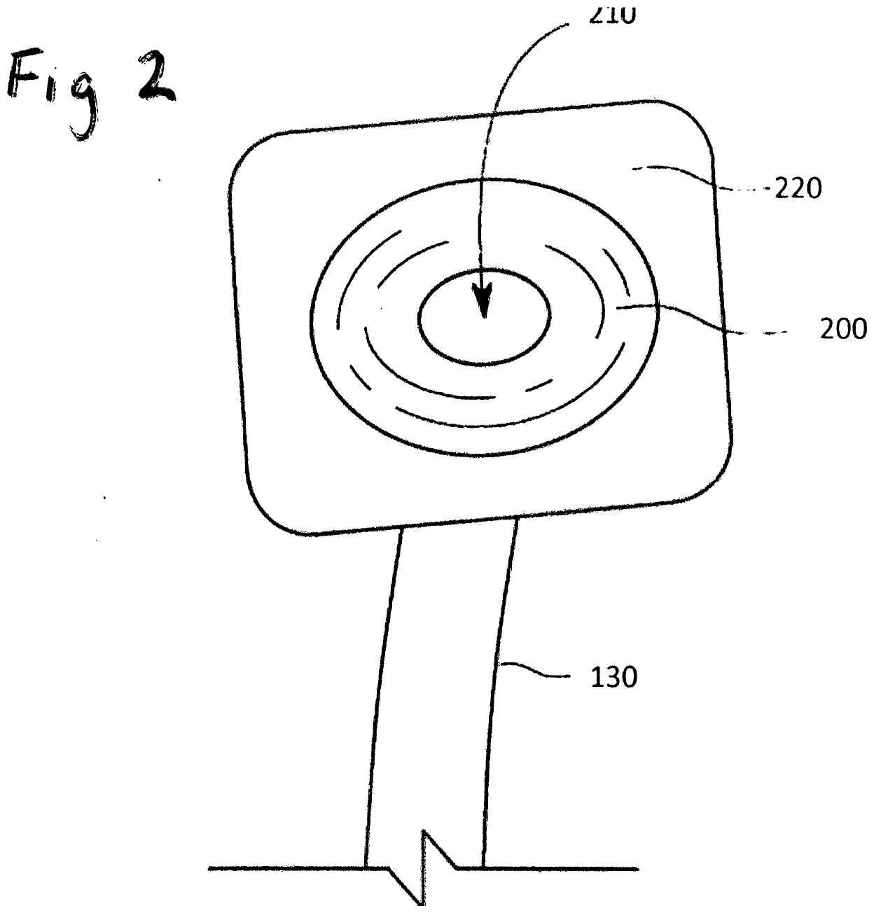

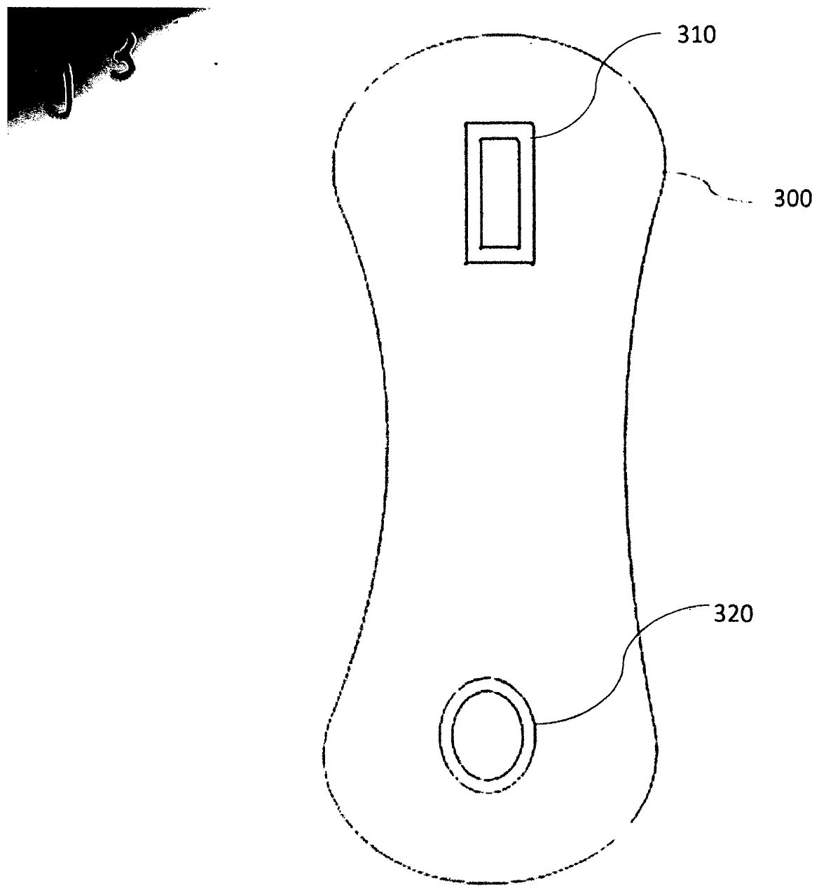

Disclosed is a body waste collection system that can receive bladder and/or bowel excretions from a person. Referring to FIG. 1, which shows an exploded view of an exemplary embodiment of the body waste collection system 100 that can include a pad 110, a tubing 120, and a waste collection bag 130. The pad 110 can be a planar or enveloping body that covers an area around a body orifice, such as the urethra or anus for collecting the excretions from the body orifice. The pad can be a urethral pad that can be applied around the labia minora for collecting urine from the urinary bladder. The pad can also be a rectum pad that can be applied around the anus to receive bowel excretions, such as feces. The pad 110 can include a central opening 140 which can juxtapose over the body orifice, such as excretions can pass through the central opening. The central opening can be shaped according to the body orifice. For example, FIG. 1 shows the pad for the urethra, wherein the central opening 140 can be an elongated rectangular shape. The central opening in a urethral pad can be rectangular, oblong, or heart shape that can juxtapose over labia minora to permit urine to pass through the central opening 140. FIG. 2 shows a rectum pad 200 that can be used to collect bowel excretions. The central opening 210 in the rectum pad 200 can be round that can juxtapose to the anus and can permit the bowel excretions to pass through. A person may need either a urethral pad or a rectum pad or both. Depending upon the requirement, the urethral pad or the rectum pad, or both can be used. The urethral pad and the rectum pad can be separately provided as shown in FIG. 1 and FIG. 2. Additionally, a single pad that has two openings for the urethra and the anus can also be provided. FIG. 3 shows such an integral pad 300 that can have a rectangular opening 310 like central opening 140 in FIG. 1 and a round opening 320 similar to the central opening 210 in FIG. 2.

The pad 110 can have a top face and a bottom face, wherein the top face can have a layer of adhesive. A layer of adhesive can be provided around a periphery of the pad 110, such as the pad can be adhered around the body orifice. The shape and size of pad 110 can be optimized for adherence to the body orifice. FIG. 2 shows the rectum pad 200 that has a layer of adhesive 220 around the periphery. The adhesive layer can be protected by a release liner, such as the user can peel off the release liner before applying the pad. Also, the pad can be planar with a layer of adhesive around a periphery. Alternatively, the pad can be enveloping that can have a flange along the periphery and the adhesive layer can be applied to the flange. The enveloping refers to having depression with a central opening at the center of the depression.

The tubing 120 can connect the central opening of the pad 110 to the waste collection bag 130. The tubing can have a proximal end and a distal end. The proximal end of the tubing can be flared to be of a shape commensurate with the shape of the central opening. For example, the flared proximal end of the tubing can be round or rectangular. A periphery of the flared proximal end can be attached around the central opening of the pad at the bottom surface, such as the excretions can pass through the central opening into the tubing which can direct the excretions to the waste collection bag 130. FIG. 1 shows the rectangular shape flared end 150 of the tubing 120. A back layer 160 can also be provided to secure the flared end 150 to the bottom surface of the pad 110. The tubing can be made of soft, flexible, and collapsible materials, such as plastic and silicon. The tubing and the flared proximal end of the tubing can be collapsed for the comfort of the person and can readily expand under the surge of urine or feces. The distal end of the tubing 120 can be removably attached to the waste collection bag 130. The waste collection bag can be removed from the tubing and replaced. Additionally, more than one waste collection bag can also be provided such as one for urine and the other for feces.

In case, both urethral and rectum pads are required by the person, two tubes can connect the two pads to one or two waste collection bags. Alternatively, a single tube can be bifurcated near the mid-portion, and two branch tubes can extend from the main tube. Each two branch tubes can include flared proximal ends, that can connect to the two pads.

The waste collection bag 130 also shown in FIG. 1 can be made by bonding two sheets along the periphery. The one or both sheets can be transparent film, such as the contents of the bag are visible to the person. One sheet can have an attachment 170 for connection with the distal end of the tubing, such as the excretions can be drained into the waste collection bag. The attachment can be an opening supported on a sheet 180. The waste collection bag 130 can also have a hanging clip 19C at edge. FIG. 1 also shows the tubing has an attachment 125 for coupling to the attachment 170 of the waste collection bag 130.

In one exemplary embodiment, the pad can be made from two sheets bonded at the edges i.e., a top sheet and a bottom sheet. The top sheet can have a central opening for the body orifice and a later of adhesive. The bottom sheet can have a second central opening that can be connected to the flared proximal end of the tubing.

In one case, the pad and the tubing can be provided as one piece, that can be reusable for a limited number of times or for single use. The tubing can also be removably and interchangeably coupled to the pad. The pad and the tubing can be manufactured in different sizes, shapes, and colors.

In one exemplary embodiment, disclosed is a method for managing bladder and/or bowel incontinence in humans and animals. Firstly, areas around the body orifices, such as the anus and urethra can be cleaned and dried. Any skin protection cream can also be applied to the skin areas. The disclosed body waste collection system can then be used by peeling the liner from the pad and applying the pad to the body orifice.

In one case, the pad can be made from hydrocolloid DuoDERM® for its many therapeutic skin properties but any soft flexible skin adhesive pad can be used without departing from the scope of the present invention. A central-opening can be made in the sheet of the pad, where the shape and size of the central opening correspond to the shape and size of the body orifice. The tubing with a flared end can be provided, wherein the flared end can be sealed around the opening. A portion of the proximal end of the tubing can pass through the central opening and then be sealed. The distal end of the tubing can then be attached to an interchangeable waste collection bag. Methods for connecting tubing to a waste collection bag are known in the art and any such method is within the scope of the present invention. A Backing layer can also be provided on the rear surface of the pad to secure the tubing to the pad. An adhesive layer can be applied around the periphery of the top face of the pad.

In one exemplary embodiment, the waste collection bag can be directly attached to the pad omitting the tubing. The opening of the waste collection bag can be sealed to the central opening of the pad, wherein the excretions are directly passed into the waste collection bag.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A body waste collection system comprising:
a pad has a top surface and a bottom surface, the pad further has a central opening of a shape and size that correspond to a shape and size of a body orifice, an adhesive layer on the top surface along a periphery;
a tubing having a proximal end and a distal end, the proximal end of the tubing is flared, wherein the flared proximal end of the tubing is of a shape and size that correspond to the shape and size of the central opening, the flared proximal end of the tubing sealably attached to a periphery of the central opening; and
a waste collection bag fluidly coupled to the distal end of the tubing.

2. The body waste collection system according to claim 1, wherein, the body orifice is a urethra, the central opening is of the shape and size configured to juxtapose to labia minora, wherein the top surface of the pad is configured to adhere to a skin area around labia minora, such as excretions from the urethra can pass through the central opening into the tubing, wherein the tubing is configured to direct the excretions into the waste collection bag.

3. The body waste collection system according to claim 2, wherein the central opening is of a rectangular shape or an oblong shape.

4. The body waste collection system according to claim 1, wherein, the body orifice is an anus, the central opening is of the shape and size configured to juxtapose to the anus, wherein the top surface of the pad is configured to adhere to a skin area around the anus, such as excretions from the anus can pass through the central opening into the tubing, wherein the tubing configured to direct the excretions into the waste collection bag.

5. The body waste collection system according to claim 4, wherein the central opening is of a round shape.

6. The body waste collection system according to claim 1, wherein the body waste collection system comprises two pads i.e., a urethral pad and a rectum pad,
wherein a central opening of the urethral pad is of a shape and size configured to juxtapose to labia minora, wherein a top surface of the urethral pad is configured to adhere to a skin area around the labia minora, such as excretions from a urethra can pass through the central opening of the urethral pad into the tubing, the tubing configured to direct the excretions into the waste collection bag,
wherein a central opening of the rectum pad is of a shape and size configured to juxtapose to an anus, wherein a top surface of the rectum pad is configured to adhere to a skin area around the anus, such as excretions from the anus can pass through the central opening of the rectum pad into the tubing, wherein the tubing is configured to direct the excretions into the waste collection bag.

7. The body waste collection system according to claim 6, wherein the tubing is bifurcated into a main tube and two branch tubes extend from the main tube, each of the two branch tubes has a flared proximal end, wherein the flared proximal end of one branch tube of the two branch tubes is configured to attach to the central opening of the rectum pad and another branch tube of the two branch tubes is configured to attach to the central opening of the urethral pad.

8. The body waste collection system according to claim 6, wherein the body waste collection system comprises two tubings and two waste collection bags, wherein one tubing and one waste collection bag is for the rectum pad, and another tubing and another waste collection bag is for the urethral pad.

9. The body waste collection system according to claim 6, wherein the body waste collection system comprises two tubings, wherein one tubing is for the rectum pad and another tubing is for the urethral pad, wherein distal ends of the two tubings attach to the waste collection bag.

10. The body waste collection system according to claim 6, wherein the two pads are integral.

11. A method for collecting bodily wastes from anus and/or urethra, the method comprising the steps of:
providing a body waste collection system comprising:
a pad has a top surface and a bottom surface, the pad further has a central opening of a shape and shape that correspond to a shape and size of a body orifice, an adhesive layer on the top surface along a periphery,
a tubing having a proximal end and a distal end, the proximal end of the tubing is flared, wherein the flared proximal end of the tubing is of a shape and size that correspond to the shape and size of the central opening, the flared proximal end of the tubing sealably attached to a periphery of the central opening, and
a waste collection bag fluidly coupled to the distal end of the tubing; and
adhering the pad to a skin area around the body orifice, such as excretions from the body orifice can pass through the central opening into the tubing.

12. The method according to claim 11, wherein the body orifice is a urethra, the central opening is of the shape and size configured to juxtapose to labia minora, wherein the top surface of the pad is configured to adhere to the skin area around the labia minora, such as excretions from the urethra can pass through the central opening into the tubing, wherein the tubing configured to direct the excretions into the waste collection bag.

13. The method according to claim 12, wherein the central opening is of a rectangular shape or an oblong shape.

14. The method according to claim 11, wherein the body orifice is an anus, the central opening is of the shape and size configured to juxtapose to the anus, wherein a top surface of the pad is configured to adhere to the skin area around the anus, such as excretions from the anus can pass through the central opening into the tubing, wherein the tubing configured to direct the excretions into the waste collection bag.

15. The method according to claim 14, wherein the central opening is of a round shape.
